(19) United States
(12) Patent Application Publication
(10) Pub. No.: US 2023/0123473 A1
(43) Pub. Date: Apr. 20, 2023

(54) DEEP BRAIN STIMULATION VIDEO PROGRAMMING PLATFORM WITH ALGORITHMS

(71) Applicant: Boston Scientific Neuromodulation Corporation, Valencia, CA (US)

(72) Inventors: Kyle Harish Srivastava, Saint Paul, MN (US); Benjamin Phillip Hahn, Austin, TX (US); Jonathan Bennett Shute, Eagan, MN (US); Amarpreet Singh Bains, Woodbury, MN (US)

(21) Appl. No.: 17/968,090

(22) Filed: Oct. 18, 2022

Related U.S. Application Data
(60) Provisional application No. 63/257,409, filed on Oct. 19, 2021.

(51) Int. Cl. A61B 5/00; G06V 40/20; G06V 40/18; G06V 10/77; A61B 5/11

(52) U.S. Cl. CPC A61B 5/4082; G06V 40/25; G06V 40/28; G06V 40/193; G06V 10/7715; A61B 5/0077; A61B 5/1128; A61N 1/36067

(57) ABSTRACT

This document discusses a computer-implemented method of machine recognition of a physiological condition of a subject. The computer-implemented method comprises obtaining a video stream of the subject using a video data source; identifying, using processing circuitry, one or more areas within image frames of the video stream that contain a physiological feature of the subject; analyzing video data of the identified one or more areas in the image frames using the processing circuitry to detect change of the physiological feature between a first frame of video data and a later frame of video data; determining one or more change parameters of the physiological feature from the video data; and generating an indication of a symptom of Parkinson's Disease according to a detection criterion applied to the one or more change parameters.

### CLAIM OF PRIORITY

This application claims the benefit of U.S. Provisional Application No. 63/257,409, filed on Oct. 19, 2021, which is hereby incorporated by reference in its entirety.

### TECHNICAL FIELD

This document relates generally to medical devices and more particularly to a system that collects and processes video data for patient diagnoses.

### BACKGROUND

Neurostimulation, also referred to as neuromodulation, has been proposed as a therapy for a number of conditions. Examples of neurostimulation include Spinal Cord Stimulation (SCS), Deep Brain Stimulation (DBS), Peripheral Nerve Stimulation (PNS), and Functional Electrical Stimulation (FES). Implantable neurostimulation systems have been applied to deliver such a therapy. An implantable neurostimulation system may include an implantable neurostimulator, also referred to as an implantable pulse generator (IPG), and one or more implantable leads each including one or more electrodes. The implantable neurostimulator delivers neurostimulation energy through one or more electrodes placed on or near a target site in the nervous system. An external programming device can be used to program the implantable neurostimulator with stimulation parameters controlling the delivery of the neurostimulation energy.

Typically, the IPG is programmed in a clinical setting by a clinician or physician to customize neurostimulation therapy to a particular patient. Remote programming of neurostimulation devices would be advantageous for the patient and clinician or physician.

### SUMMARY

In DBS, electrical neurostimulation therapy is delivered from an IPG to implantable electrodes located at certain neurostimulation targets in the brain to treat neurological or neurophysiological disorders of a patient or subject. The IPG is programmed to customize neurostimulation therapy for a particular patient. Analysis of video of the patient allows for algorithms performed by a medical device to remotely assess treatment of the patient. The assessment can be used to improve the patient's therapy.

Example 1 includes subject matter (such as a computer-implemented method of machine recognition of a physiological condition of a subject) comprising obtaining a video stream of the subject using a video data source, identifying, using processing circuitry, one or more areas within image frames of the video stream that contain a physiological feature of the subject, analyzing video data of the identified one or more areas in the image frames using the processing circuitry to detect change of the physiological feature between a first frame of video data and a later frame of video data, and determining one or more change parameters of the physiological feature from the video data.

In Example 2, the subject matter of Example 1 optionally includes analyzing the video data of the identified areas in the image frames using the processing circuitry to detect movement of the physiological feature between a first frame of video data and a later frame of video data, determining one or more movement parameters of the physiological feature from the video data, and generating an indication of a symptom of Parkinson's Disease according to a detection criterion applied to the one or more movement parameters.

In Example 3, the subject matter of Example 2 optionally includes identifying one or more areas within the image frames that contain at least one of a hand or arm of the subject, determining one or both of amplitude and frequency of movement of the at least one of the hand or arm, and generating an indication of a tremor of the subject according to the determined one or both of amplitude and frequency.

In Example 4, the subject matter of Example 3 optionally includes changing, by the processing circuitry, a parameter of neurostimulation provided to the subject according to the determined one or both of amplitude and frequency of movement.

In Example 5, the subject matter of Example 4 optionally includes changing, by the processing circuitry, one or both of a target amplitude and a target frequency bandwidth of movement of the at least one of the hand or arm.

In Example 6, the subject matter of one or any combination of Examples 2-5 optionally includes identifying an area within the image frames that contains eyes of the subject, determining frequency of eye blinking of the subject, and generating an indication of a change in the frequency of the eye blinking as the symptom of Parkinson's disease.

In Example 7, the subject matter of one or any combination of Examples 2-6 optionally includes identifying an area within the image frames that contains eyes of the subject, detecting saccadic eye movement of the subject, and generating an indication of abnormal eye movement as the symptom of Parkinson's disease.

In Example 8, the subject matter of Example 7 optionally includes changing, by the processing circuitry, a parameter of neurostimulation provided to the subject in response to the detected abnormal eye movement.

In Example 9, the subject matter of one or any combination of Examples 2-8 optionally includes identifying one or more areas within the image frames that contain at least one limb of the subject, determining one or more of limb position and direction of limb movement of the at least one limb, and generating an indication of abnormality in gait of the subject according to one or more detection criteria applied to the determined one or more of limb position and direction of limb movement.

In Example 10, the subject matter of Example 9 optionally includes changing, by the processing circuitry, a parameter of neurostimulation provided to the subject in response to the detected gait abnormality.

In Example 11, the subject matter of one or any combination of Examples 2-10 optionally includes identifying an area within the image frames that contains a thorax of the subject, determining one or both of amplitude and frequency of movement of the thorax, and generating an indication of change in respiratory rate of the subject according to the determined one or both of amplitude and frequency.

In Example 12, the subject matter of one or nay combination of Examples 2-11 optionally includes identifying an area within the image frames that contain a facial image of the subject, analyzing the video data of the facial image to detect a change in appearance of the facial image between a first frame of video data and a later frame of video data, detecting one or more of facial rigidity, a change in pupil dilation, and a change in skin pigment, and generating an indication of a symptom of Parkinson's Disease according to a detection criterion applied to the one or more change parameters.

In Example 13, the subject matter of Example 12 optionally includes changing, by the processing circuitry, a parameter of neurostimulation provided to the subject in response to the detected change in one or more of facial rigidity, pupil dilation, and skin pigment.

In Example 14, the subject matter of one or any combination of Examples 1-13 optionally includes obtaining a video stream of the subject using a video data source includes obtaining the video stream using a device to provide prompts to a user while obtaining the video stream.

Example 15 includes subject matter (such as an electronic device) or can optionally be combined with one or any combination of Examples 1-14 to include such subject matter, comprising a video data source configured to obtain a video stream that includes image frames of video data, processing circuitry, and a memory. The memory is to store instructions that, when performed by the processing circuitry, cause the processing circuitry to perform operations including identifying one or more areas within the image frames of the video stream that contain a physiological feature of a subject, analyzing video data of the identified one or more areas in the image frames to detect movement of the physiological feature between a first frame of video data and a later frame of video data, determining one or more movement parameters of the at least one physiological feature from the video data, and generating an indication of a tremor associated with

Parkinson's Disease according to a detection criterion applied to the one or more movement parameters.

In Example 16, the subject matter of Example 15 optionally includes instructions that cause the processing circuitry to perform operations including identifying one or more areas within the image frames that contain at least one of a hand or arm of the subject, determining one or both of amplitude and frequency of movement of the at least one of the hand or arm, and generating an indication of a tremor of the subject according to the determined one or both of amplitude and frequency.

In Example 17, the subject matter of one or both of Examples 15 and 16 optionally includes instructions that cause the processing circuitry to perform operations including identifying one or more areas within the image frames that contain at least one limb of the subject, determining one or more of limb position and direction of limb movement of the at least one limb, and generating an indication of abnormality in gait of the subject according to one or more detection criteria applied to the determined one or more of limb position and direction of limb movement.

In Example 18, the subject matter of one or both of Examples 15-17 optionally includes instructions that cause the processing circuitry to change one or more parameters of neurostimulation therapy provided to the subject by a separate device according to the determined one or more movement parameters.

Example 19 includes subject matter (such as an electronic device) or can optionally be combined with one or any combination of Examples 1-18 to include such subject matter, comprising a video data source configured to obtain a video stream that includes image frames of video data, processing circuitry, and a memory. The memory is to store instructions that, when performed by the processing circuitry, cause the processing circuitry to perform operations including identifying one or more areas within the image frames of the video stream that contain a facial image of a subject, analyzing video data of the identified one or more areas in the image frames to detect a change in appearance of the facial image between a first frame of video data and a later frame of video data, detecting one or more of facial rigidity, a change in pupil dilation, and a change in skin pigment from the video data, and generating an indication of a symptom of Parkinson's Disease according to a detection criterion applied to the detected one or more of facial rigidity, change in pupil dilation, and change in skin pigment.

In Example 20, the subject matter of Example 19 optionally includes instructions that cause the processing circuitry to change one or more parameters of neurostimulation therapy provided to the subject by a separate device according to the detected change in the one or more of facial rigidity, pupil dilation, and skin pigment.

These non-limiting examples can be combined in any permutation or combination. This summary is intended to provide an overview of subject matter of the present patent application. It is not intended to provide an exclusive or exhaustive explanation of the disclosure. The detailed description is included to provide further information about the present patent application. Other aspects of the disclosure will be apparent to persons skilled in the art upon reading and understanding the following detailed description and viewing the drawings that form a part thereof, each of which are not to be taken in a limiting sense.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is an illustration of portions of an example of an electrical stimulation system.

FIGS. 2A-2C illustrate additional examples of electrical stimulation systems.

FIG. 3 is a flow diagram of a computer-implemented method of machine recognition of a physiological condition of a patient or subject.

FIG. 4 is an illustration of a system to implement an example of the method of FIG. 3.

FIG. 5 is a flow diagram of an example of a method of using a live video feed of a subject to enable live tracking of the physiological response of the subject to different neurostimulation programming settings.

FIGS. 6 and 7 are further illustrations of FIG. 6 examples of the method of FIG. 3.

### DETAILED DESCRIPTION

In the following detailed description, reference is made to the accompanying drawings which form a part hereof, and in which is shown by way of illustration specific embodiments in which the invention may be practiced. These embodiments are described in sufficient detail to enable those skilled in the art to practice the invention, and it is to be understood that the embodiments may be combined, or that other embodiments may be utilized, and that structural, logical and electrical changes may be made without departing from the spirit and scope of the present invention. References to “an”, “one”, or “various” embodiments in this disclosure are not necessarily to the same embodiment, and such references contemplate more than one embodiment. The following detailed description provides examples, and the scope of the present invention is defined by the appended claims and their legal equivalents.

This document discusses devices, systems and methods for programming and delivering electrical neurostimulation to a patient or subject. Advancements in neuroscience and neurostimulation research have led to a demand for delivering complex patterns of neurostimulation energy for various types of therapies. The present system may be implemented using a combination of hardware and software designed to apply any neurostimulation (neuromodulation) therapy, including but not being limited to DBS therapy.

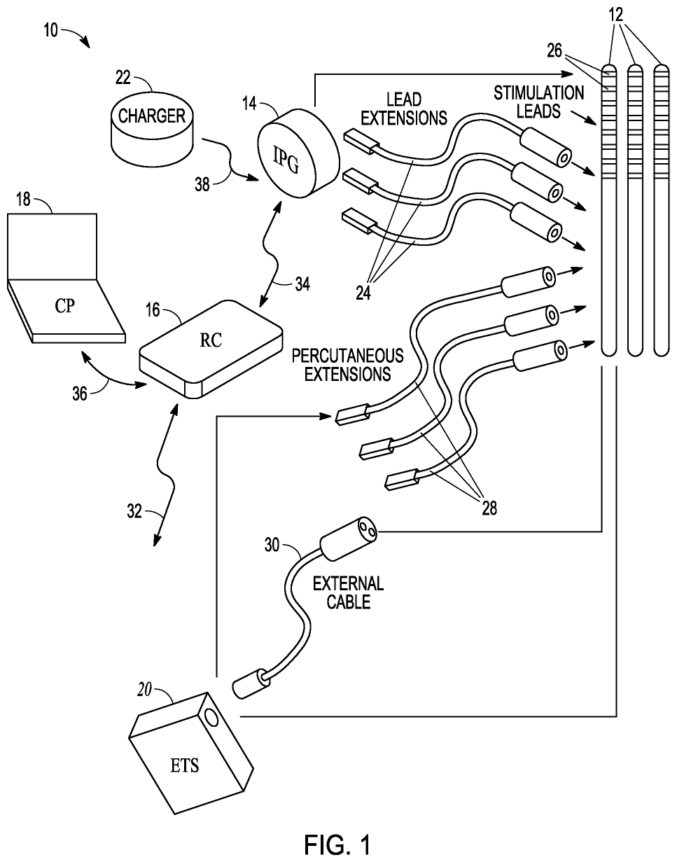

FIG. 1 is an illustration of portions of an embodiment of an electrical stimulation system 10 includes one or more stimulation leads 12 and an implantable pulse generator (IPG) 14. The system 10 can also include one or more of an external remote control (RC) 16, a clinician's programmer (CP) 18, an external trial stimulator (ETS) 20, or an external charger 22. The IPG 14 can optionally be physically connected via one or more lead extensions 24, to the stimulation lead(s) 12. Each lead carries multiple electrodes 26 arranged in an array. The IPG 14 includes pulse generation circuitry that delivers electrical stimulation energy in the form of, for example, a pulsed electrical waveform (i.e., a temporal series of electrical pulses) to the electrode array 26 in accordance with a set of stimulation parameters. The IPG 14 can be implanted into a patient's body, for example, below the patient's clavicle area or within the patient's buttocks or abdominal cavity. The implantable pulse generator can have multiple stimulation channels (e.g., 8 or 16) which may be independently programmable to control the magnitude of the current stimulus from each channel. The IPG 14 can have one, two, three, four, or more connector ports, for receiving the terminals of the leads 12.

The ETS 20 may also be physically connected, optionally via the percutaneous lead extensions 28 and external cable 30, to the stimulation leads 12. The ETS 20, which may have similar pulse generation circuitry as the IPG 14, can also deliver electrical stimulation energy in the form of, for example, a pulsed electrical waveform to the electrode array 26 in accordance with a set of stimulation parameters. One difference between the ETS 20 and the IPG 14 is that the ETS 20 is often a non-implantable device that is used on a trial basis after the neurostimulation leads 12 have been implanted and prior to implantation of the IPG 14, to test the responsiveness of the stimulation that is to be provided. Any functions described herein with respect to the IPG 14 can likewise be performed with respect to the ETS 20.

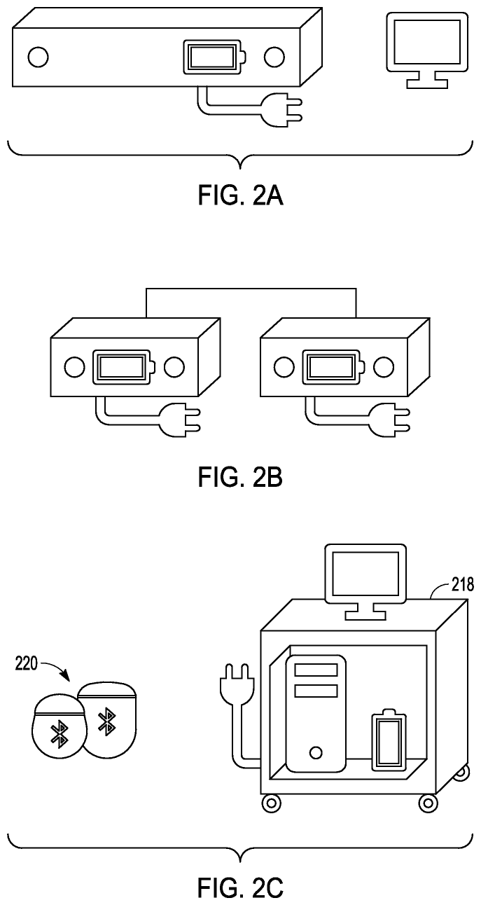

FIGS. 2A-2C illustrate examples of additional hardware configurations. In FIG. 2A, the electrical stimulation hardware, the sensing hardware, and a user interface are included in the same unit. In FIG. 2B, the electrical stimulation hardware and the sensing hardware are provided as separate units. The unit or units of FIGS. 2A and 2B can be capital equipment and can be rack mounted. The units shown in FIGS. 2A and 2B are wall powered but the units can be battery powered. FIG. 2C illustrates an example that includes an ETS 220 and separate computer 218. The computer 218 communicates wirelessly with the ETS 220 (e.g., using Bluetooth hardware and communication protocol).

Returning to FIG. 1, the RC 16 may be used to telemetrically communicate with or control the IPG 14 or ETS 20 via a wireless communications link 32. Once the IPG 14 and neurostimulation leads 12 are implanted, the RC 16 may be used to telemetrically communicate with or control the IPG 14 via communications link 34. The communication or control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. The CP 18 allows a user, such as a clinician, the ability to program stimulation parameters for the IPG 14 and ETS 20 in the operating room and in follow-up sessions. The CP 18 may perform this function by indirectly communicating with the IPG 14 or ETS 20, through the RC 16, via a wireless communications link 36. Alternatively, the CP 18 may directly communicate with the IPG 14 or ETS 20 via a wireless communications link (not shown). The stimulation parameters provided by the CP 18 are also used to program the RC 16, so that the stimulation parameters can be subsequently modified by operation of the RC 16 in a stand-alone mode (i.e., without the assistance of the CP 18).

For purposes of brevity, the details of the RC 16, CP 18, ETS 20, and external charger 22 will not be further described herein. Details of exemplary embodiments of these devices are disclosed in U.S. Pat. No. 6,895,280, which is incorporated herein by reference. Other embodiments of electrical stimulation systems can be found at U.S. Pat. Nos. 6,181,969; 6,516,227; 6,609,029; 6,609,032; 6,741,892; 7,949,395; 7,244,150; 7,672,734; 7,761,165; 7,974,706; 8,175,710; 8,224,450; 8,364,278; and 8,700,178, all of which are incorporated herein by reference.

Remote device-based analysis of the patient's symptoms and programming of devices for delivering neurostimulation therapy such as DBS would be desirable. Video data obtained using a camera included in a smartphone or computer located with the patient can be sent to another computing device (e.g., in the cloud) to enable algorithms to be performed on the video data to assess the patient's condition. In some examples, the algorithm or algorithms that assess the patient's condition are performed using the smartphone or computer that includes the camera, and results of the assessment are sent to a physician or clinician. Results of the analysis can be used to program any of the external RC 16, CP 18, or ETS 20. The programming may be performed by the device providing the assessment or by the physician or clinician.

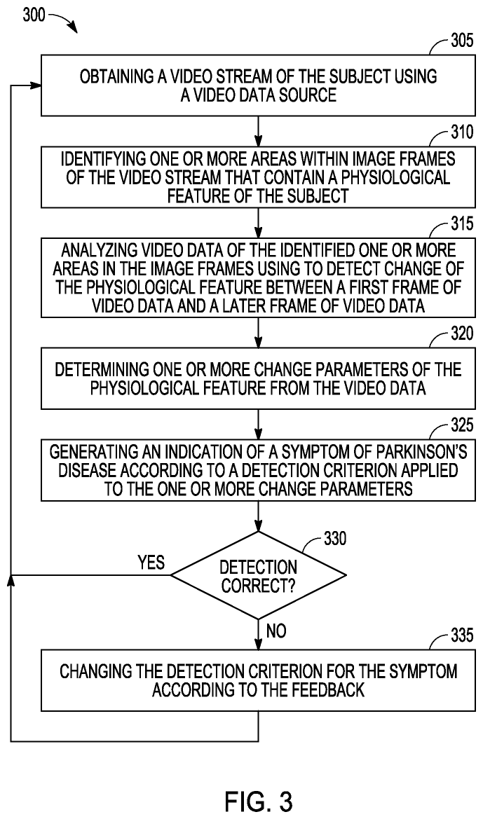

FIG. 3 is a flow diagram of a computer-implemented method 300 of machine recognition of a physiological condition of a patient or subject. At block 305, a video stream of the subject is obtained using a video data source. An example of the video data source is a camera. The camera may be included in a computer (e.g., laptop computer, desktop computer, tablet computer, etc.) or smartphone. The video stream includes image frames of video data. In certain examples, the video data source is a memory storing the image frames of video data.

At block 310, the video data is analyzed using processing circuitry (e.g., one or more hardware processors) of the computing device implementing the method. The processing circuitry identifies one or more areas within the image frames that include a physiological feature of the subject (e.g., face, hands, arm, etc.). At block 315, the video data containing the physiological feature is analyzed by the processing circuitry to detect a change in the physiological feature between image frames of the video stream. The change may be the appearance of the physiological feature or a change in position of the physiological feature associated with movement of the subject. The change may be related to a symptom of the patient.

At block 320, the processing circuitry quantifies the change in the physiological feature by determining one or more change parameters of the physiological feature. At block 325, the processing circuitry applies one or more detection criteria for the physiological condition to the one or more change parameters. If the condition is detected using the criteria, an indication of the physiological condition is generated which can be presented to a physician or provided to another process. In some examples, the detection criteria are used to detect a symptom related to Parkinson's Disease, and an indication of the symptom associated with Parkinson's Disease is generated.

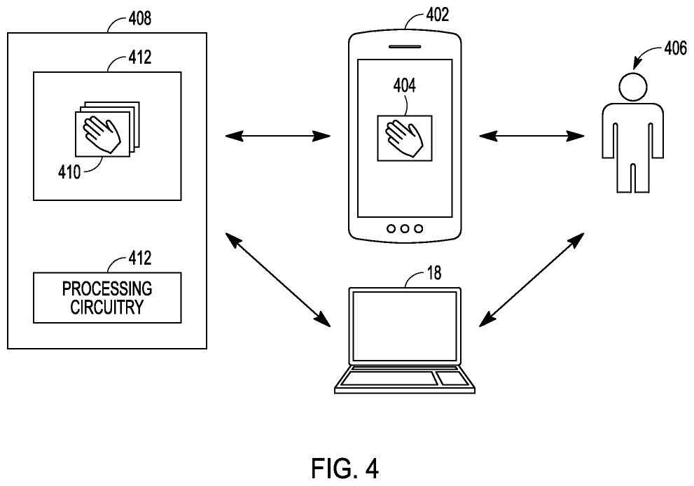

FIG. 4 is an illustration of a system to implement an example of the method 300 of FIG. 3. A smartphone 402 is used to obtain video data 404 of the patient 406. The video data 404 is sent to a computing device 408 for analysis. The computing device 408 may be remote from the patient 406 and smartphone 402. The computing device 408 may be a computer, another smartphone, or a server.

The processing circuitry 412 of the computing device 408 analyzes video data 404 of one or both of the hand or arm of the patient to detect motor tremors due to Parkinson's Disease. In the example of FIG. 4, image frames 410 that include a hand of the patient are stored in memory 414 of the computing device 408 and analyzed. To obtain the video data 404, a user (e.g., clinician or physician) of the computing device 408 may ask (via the smartphone) the patient to place their hand in view of the camera. The video stream taken by the camera is sent to the computing device 408. In certain embodiments, a user of the computing device 408 is not needed, and one or more prompts are provided to the smartphone to prompt the patient to place one or both hands in view of the camera. The prompts may be sent to the smartphone by the computing device 408 (e.g., via a cellular network or the Internet) or the prompts may be included in an application (or App) running on the smartphone. In some examples, the processing circuitry 412 uses object detection to identify the area in the image frames of the video stream that contain the image of the hand of the patient. In certain examples, the processing circuitry 412 detects the image of the hand without a prompt to the patient or having the patient position their hand in front of the camera.

Using the video data, the processing circuitry 412 identifies key points of the hand and detects movement of the hand between image frames and calculates one or more movement parameters from the image frames. In some examples, the movement parameters can include one or both of the amplitude of the movement of the hand and the frequency of the movement of the hand. The processing circuitry 412 applies a detection criterion to the determined amplitude and frequency to detect tremors of the subject. The detection criterion may detect that the movement of the hand has an amplitude that exceeds a predetermined amplitude threshold, and the movement is within a frequency range associated with tremors.

If detected, the processing circuitry 412 generates an indication of a tremor or tremors. For instance, the computing device 408 may include a display and the computing device 408 presents an indication using the display to the user that the patient is experiencing tremors. In some examples, the computing device sends the indication of tremors to a CP 18. The clinician or physician may change one or more parameters of neurostimulation therapy provided to the patient to reduce patient tremors.

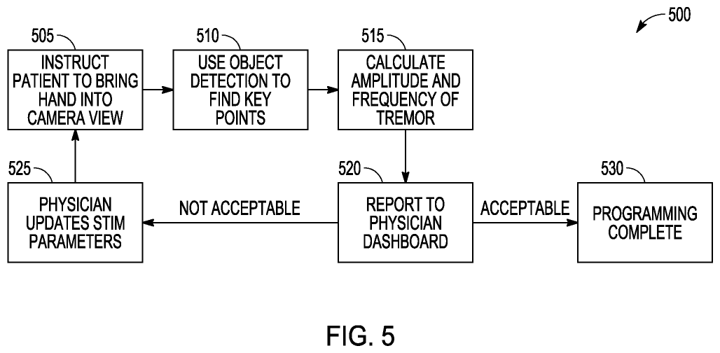

FIG. 5 is a flow diagram of an example of a method 500 of using a live video feed to enable live tracking of the physiological response of the patient to different neurostimulation programming settings. The method 500 may be implemented using the computing device 408 of FIG. 4. A physician can communicate with the patient 406 via a smartphone 402 or a patient computer. At block 505, the physician instructs the patient to bring their hand into the view of the camera. In certain examples, the computing device 408 provides a prompt on a display screen as a target and the patient move their hand toward the prompt. In certain examples, the prompt is an area of the display screen, or an outline of a hand.

At block 510, the computing device 408 uses object detection to find key points of the hand. At block 515, the computing device 408 calculates one or both of the amplitude and frequency of movement of the patient's hand. At block 520, an indication of a tremor is generated and reported to the physician when a tremor is detected. The tremor may be reported to the physician (on a displayed physician dashboard) using the computing device 408 or CP 18. The amplitude and frequency of the tremor may be reported or an indication of strength of the tremor may be reported.

If the physician feels the tremors are unacceptably strong, at block 525, the physician updates one more of the parameters of the neurostimulation (e.g., DBS). The computing device 408 may continue with reporting of the tremors. If the physician feels the tremors are acceptable or have disappeared, at block 530, the programming of the neurostimulation parameters is completed.

Returning to FIG. 4, in some examples, the computing device 408 suggests changes to one or more neurostimulation therapy parameters using a display of the computing device 408 or a display of the CP 18. The one or more neurostimulation therapy parameters can include changing one or more of the amplitude of neurostimulation pulses, the frequency of the neurostimulation pulses, and the location of the neurostimulation pulses. The functions described may be divided between the computing device 408 and the smartphone 402. Some of the functions attributed to processing circuitry 412 of the computing device 408 may be performed by processing circuitry of the smartphone 402.

Returning to FIG. 3, at block 330 the computing device can receive feedback as to the accuracy of the detection of the physiological condition or conditions by the computing device. If the feedback is that the detection was not accurate, at block 335, the computing device changes the detection criteria for the detection of the physiological condition. The method 300 then returns to block 305 to continue detection. For instance, in the example of FIG. 4, the clinician or physician may provide feedback of accuracy of the detection of tremors using a user interface of the computing device 408 or the CP 18. The computing device 408 changes the detection criteria to improve the algorithm for detection of tremors, such as by changing an amplitude and frequency analysis (e.g., a Fast Fourier Transform analysis) performed by the processing circuitry. Returning to FIG. 3, if the feedback at block 330 is that the detection was accurate, the method 300 returns to block 305 and the detection may continue as before.

In some examples, the computing uses machine learning to adjust the detection criteria. Machine learning (e.g., supervised machine learning) may be used to train a neural network model (a neural model implemented using instructions stored in memory that cause the processing circuitry to perform the neural network model). The processing circuitry 412 may retrieve and use the neural network model when making an adjustment to the detection criteria. In an example where a neural network model is used to adjust the detection algorithm, the number of adjustments to improve accuracy of detection may be reduced compared to the case where no neural network model is used.

In some examples, the processing circuitry 412 may change one or more neurostimulation parameters in response to detection of the physiological condition. For example, machine learning may be used to train the neural network to adjust one or more neurostimulation parameters based on detecting tremors of the patient. The parameters are changed by the neural network with the goal of reducing tremors. The changed parameters may be transmitted to the CP 18 which may change the neurostimulation therapy of an IPG 14. In an example where a neural network model is used to adjust the one or more neurostimulation parameters, the number of adjustments may be reduced compared to the case where no neural network model is used. In some examples, the processing circuitry changes one or both of a target amplitude and a target frequency bandwidth of movement for the neurostimulation.

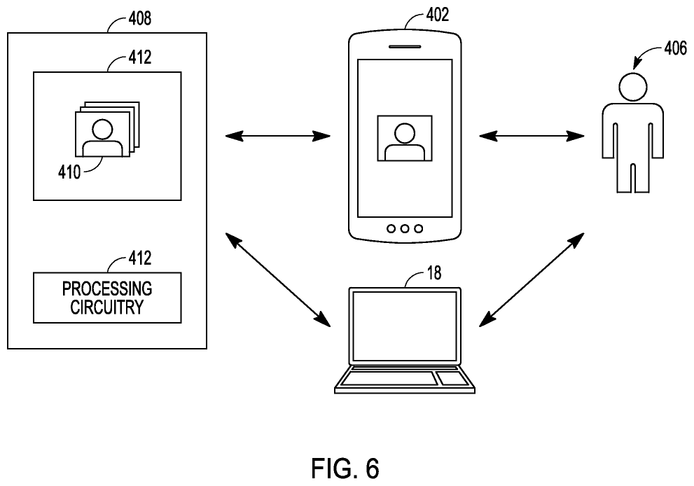

FIG. 6 is an illustration of another example of the method 300 of FIG. 3. The processing circuitry 412 of the computing device 408 analyzes video data 410 of image frames that contain one or both eyes of the patient to detect symptoms related to Parkinson's Disease.

According to some examples, the processing circuitry 412 identifies an image of one or both of the eyes of the patient in the image frames of the video stream and detects blinking of the eye or eyes between image frames. The processing circuitry 412 calculates one or more parameters related to the blinking of the eyes detected in the image frames. In some examples, the one or more parameters include the frequency of eye blinking by the subject. The processing circuitry 412 applies a detection criterion to the determined frequency to detect whether the eye blinking indicates Parkinson's Disease. The detection criterion may detect that the frequency of blinking is within a frequency range associated with Parkinson's Disease. In some examples, the processing circuitry 412 uses one or both of detected tremors and frequency of eye blinking as feedback used to adjust neurostimulation therapy. In some examples, the processing circuitry 412 receives feedback of the accuracy of its indication of Parkinson's Disease. If the detection is inaccurate, the processing circuitry 412 may change the eye blinking detection frequency range.

According to some examples, the processing circuitry 412 identifies an image of the eyes of the patient in the image frames and detects saccadic eye movement in the video data. Healthy individuals use saccades to move their eyes onto a new target. In patients with deficient saccades, the eyes may exhibit greater positional errors during saccades or longer durations for the saccade. The smartphone 402 (or patient's computer) may provide a prompt to the user to follow a target displayed using the smartphone 402. The smartphone 402 may include an App to provide this function. In some examples, video data that includes the target to track is sent to the smartphone 402 or to a patient computer.

If deficiency in saccadic eye movement is detected, the processing circuitry 412 may generate an indication of abnormal eye movement as the symptom associated with Parkinson's Disease. In some examples, the processing circuitry 412 uses one or more of tremors, frequency of eye blinking, or abnormal saccadic eye movement as feedback used to adjust neurostimulation therapy. If the processing circuitry 412 receives feedback that the detection of deficiency in saccadic eye movement is inaccurate, the processing circuitry 412 may change the algorithm for detection of abnormal saccadic eye movement (e.g., change an eye movement latency detection threshold).

According to some examples, the processing circuitry 412 identifies a facial image of the patient in the image frames and analyzes the video data of the facial image to detect a change in appearance of the facial image between a first frame of video data and a later frame of video data. The image frames may be obtained during the same video session or obtained in different video sessions. The analysis by the processing circuitry 412 may look to detect a change in one or more of facial rigidity of the patient, a change in pupil dilation, and a change in skin pigment of the subject.

To detect facial rigidity, the processing circuitry 412 may identify key points in the facial image and calculate the variability in the distance between the key points. Variability, or a change in the determined variability, may indicate a symptom of Parkinson's Disease. The analysis of key points in the facial image by the processing circuitry 412 may also detect a change in the patient's ability to smile; indicating a symptom of Parkinson's Disease.

To monitor pupil dilation, the processing circuitry may measure the size of the patient's pupils in the facial image. The pupil size may be measured passively with the light in the room of the patient, or a light source of the video platform of the computer or smartphone of the patient may provide a light source for the measurement. A change in the ability of the pupils to contract may be detected as a change in the amount of contraction or a change in the time needed for pupil contraction. Pupil dilation or a change in pupil dilation may be a symptom of Parkinson's Disease.

To detect skin flushing, the processing circuitry 412 may store a baseline facial image and detect a change in skin color between the present facial image and the baseline facial image. The processing circuitry 412 may generate an indication of a symptom of Parkinson's Disease in response to a detected change in one or more of in facial rigidity of the patient, pupil dilation, and skin pigment of the patient. In response to feedback of the accuracy of the detection, the processing circuitry may change one or more detection criteria. For example, the processing circuitry 412 may change the threshold variability in facial key points for detection of facial rigidity, or the threshold change in pupil size to detect a change in pupil contraction. In some examples, the processing circuitry 412 uses one or more of tremors, frequency of eye blinking, saccadic eye movement, and change in facial appearance as feedback used to adjust neurostimulation therapy.

According to some examples, the processing circuitry 412 analyzes the video data of the patient's facial image to determine heart rate and heart variability. The processing circuitry may use Eulerian video magnification to detect blood flow in the image frames. The Eulerian video magnification takes video stream of the facial image as input and amplifies the processed video data to detect blood flow as it fills the face of the patient. The blood flow may be detected from amplified small facial movements due to blood flow or amplified changes in pigmentation due to blood flow. The patient's pulse or heartbeats are related to the blood flow and can be determined using the detected blood flow. The processing circuitry 412 can determine average heart rate and heart rate variability from the heartbeats. Heart rate variability may decrease in persons with Parkinson's Disease, and monitoring heart rate variability can help detect Parkinson's Disease or monitor progression of the disease. The processing circuitry 412 may generate an alert to bring attention to a detected change in heart variability.

According to some examples, the processing circuitry 412 identifies an image of the patient's thorax region in the image frames and analyzes the video data of the patient's thorax to detect respiratory rate of the patient. The processing circuitry 412 may determine respiratory rate by detecting movement of the patient's thorax and determining one or both of amplitude and frequency of the movement. A change in the respiratory rate may indicate a change in the respiratory volume of the patient. The processing circuitry 412 may generate an indication of a symptom of Parkinson's Disease in response to a detected change in the respiratory rate. If the processing circuitry 412 receives feedback that the detection is not indicative of a symptom of Parkinson's Disease, the processing circuitry 412 may change one or both of the respiratory detection amplitude and the respiratory detection frequency used to generate the indication.

Heart rate, heart rate variability, and respiratory rate can be indicative of autonomic function, which is a key aspect of non-motor symptoms of Parkinson's Disease. The processing circuitry 412 may generate an alert related to Parkinson's Disease using one or a combination of heart rate, heart rate variability, and respiratory rate.

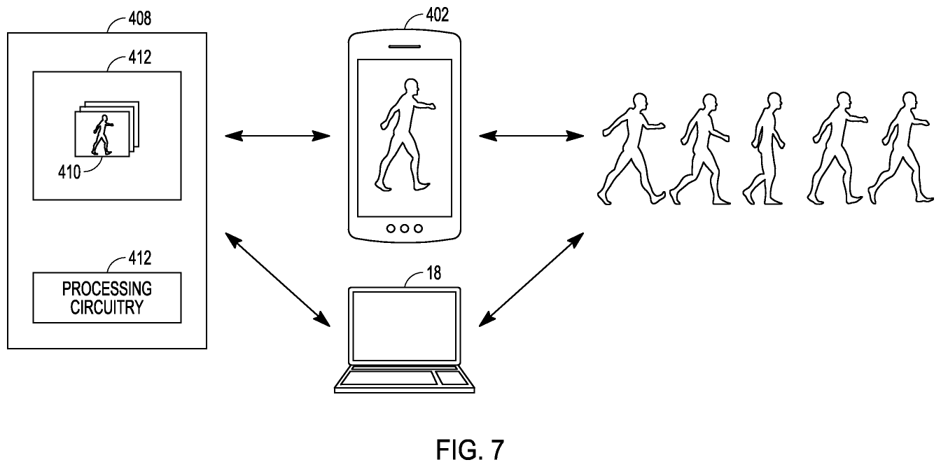

FIG. 7 is an illustration of another example of the method 300 of FIG. 3. Walking is complicated by Parkinson's Disease. A person with the disease may experience abnormalities in their gait or freezing at certain points of their gait cycle. The processing circuitry 412 of the computing device 408 analyzes video data 410 of image frames to identify one or more areas within the image frames that contain a limb or limbs of the patient, and analyzes the video data to detect a change in gait of the patient. The video stream may include one or more gait cycles of the patient. The processing circuitry 412 determines one or more of limb position, direction of limb movement, and patient posture, and detects abnormality in the patient's gait. The processing circuitry 412 may generate an indication of a symptom of Parkinson's Disease in response to a detected gait abnormality. If the processing circuitry 412 receives feedback that the detection is not indicative of a symptom of Parkinson's Disease, the processing circuitry may change one more criterion for abnormal gait detection.

Neurostimulation may help improve gait of the patient. Specific DBS patterns, delivered at specific times, may help reduce abnormity in gait. The computing device 408 may provide a live video feed of the patient walking to a physician to enable live tracking of the physiological response of the patient to different neurostimulation programming. In some examples, machine learning is used by the processing circuitry 412 to adjust one or more neurostimulation parameters to improve the gait of the patient. The processing circuitry 412 may compare the gait in the video data to a gait pattern that is average or normal for a person of the demographic of the patient. The processing circuitry 412 uses machine learning to adjust neurostimulation parameters to reduce differences in the patient gait from the normal gait pattern.

Changes to different aspects of a patient's motor movement may be detected using prompts provided using the smartphone 402 or the patient's computer. For instance, the patient's computer or smartphone 402 may include an App to provide a game that the patient can play offline or online. Key metrics determined from the interaction with the App are uploaded to the cloud when patient is back online. Motor tremor behaviors can be later tracked using the data collected by the App. For example, using a stylus, the game might be to do a maze or crossword puzzle, where the smoothness of the patient's writing and cognitive abilities are evaluated by algorithms of the App in the background.

Patients with Parkinson's Disease can demonstrate changes in their impulse control with DBS. An App may be used to quantify the impulse control of the patient. In an example, the App may include a casino game like blackjack or poker. In another example, the App may include a “Deal or No Deal” game simulation in which the patient must choose between a sure thing and a risk to gain more. The App provides prompts to a user that invites risk-taking by the user and quantifies the risk in responses to the prompts by the user. The App quantifies the risk that the patient is willing to take during the playing of the games. This is different from tracking success of the patient in the games. For instance, a risk tolerance score can be determined for the patient from the standard deviation of outcomes when the patient chooses to avoid risk. The device-quantified risk can be stored as the patient uses the App and the results can be later uploaded by the clinician or physician. Changes in the device-quantified risk may be a symptom of Parkinson's Disease.

In some examples, changes in writing by the patient are monitored. The App may have the patient draw a spiral on a piece of paper or the patient's tablet computer. The results are sent by video to the computing device 408 by the computer or smartphone for assessment of accuracy, or the accuracy of writing is assessed using an App on the computer or smartphone 402.

The computing device 408 may provide a live video feed of the patient writing to a physician to enable live tracking of the physiological response of the patient to different neurostimulation programming. One or more parameters of the neurostimulation may be adjusted to improve the writing. In some examples, machine learning is used by the processing circuitry 412 of the computing device 408 to adjust one or more neurostimulation parameters to reduce motor tremors and improve the ability of the patient to write.

Speech can be complicated by Parkinson's Disease. A person with the disease may experience abnormalities in their speech. In some examples, the processing circuitry 412 provides remote monitoring of the patient's speech. The patient speaks into the smartphone 402 and live speech is relayed to the clinician or physician. The clinician or physician can remotely change neurostimulation parameters to minimize speech errors. In some examples, the processing circuitry 412 uses machine learning to automatically reprogram neurostimulation parameters to minimize speech errors.

Speech data can also be used to monitor emotions of the patient. Emotional regulation can be impaired for patients with Parkinson's Disease. Speech data can be monitored to derive emotional content of the patient's speech. The emotional content can identify patients with emotional regulation challenges. Trending speech data over time can be used to assess likelihood of depression or emotional dysregulation. Neurostimulation settings can be titrated over time to improve the patient's emotional regulation.

A video or audio diary of the patient can be recorded. An App of the patient's computer or smartphone can include an App to provide prompts to the patient for the diary. For example, the App may include a chatbot that provides prompts to the patient such as “tell me about your day.” The patient's responses can be recorded in the diary. Further prompts may ask the patient “were there any activities today that were particularly challenging?” Prompts from the chatbot may be changed randomly and the patient can start and stop the diary as they desire.

This reporting feature can be used by the patient to report a change in symptoms or a side effect of neurostimulation therapy. Reports of side effects or other unexpected motor behaviors may be used as feedback to change detection criteria or neurostimulation programming parameters. Video can be used to record the symptom to the extent it is visible, or to record the patient's description of the change or side effect. The App may include a splash screen to inform the patient to seek immediate medical treatment if the patient is experiencing an emergency.

The devices, systems and methods described herein provide more information to clinicians and physicians regarding a physiological condition of patients, especially patients with Parkinson's Disease. The information can be collected without visits to a clinic. In many cases described, the information can be collected passively without participation of a clinician or physician. Device-based collection and processing of the information can improve care for the patients.

The embodiments described herein can be methods that are machine or computer-implemented at least in part. Some embodiments may include a computer-readable medium or machine-readable medium encoded with instructions operable to configure an electronic device or system to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. Various embodiments are illustrated in the figures above. One or more features from one or more of these embodiments may be combined to form other embodiments.

The above detailed description is intended to be illustrative, and not restrictive. The scope of the disclosure should, therefore, be determined with references to the appended claims, along with the full scope of equivalents to which such claims are entitled.

What is claimed is:

1. A computer-implemented method of machine recognition of a physiological condition of a subject, the method comprising:
obtaining a video stream of the subject using a video data source;
identifying, using processing circuitry, one or more areas within image frames of the video stream that contain a physiological feature of the subject;
analyzing video data of the identified one or more areas in the image frames using the processing circuitry to detect change of the physiological feature between a first frame of video data and a later frame of video data;
determining one or more change parameters of the physiological feature from the video data; and
generating an indication of a symptom of Parkinson's Disease according to a detection criterion applied to the one or more change parameters.

2. The method of claim 1,
wherein the analyzing the video data includes analyzing the video data of the identified areas in the image frames using the processing circuitry to detect movement of the physiological feature between a first frame of video data and a later frame of video data;
wherein the determining the one or more change parameters includes determining one or more movement parameters of the physiological feature from the video data; and
wherein the generating the indication of a symptom of Parkinson's disease includes generating an indication of a symptom of Parkinson's Disease according to a detection criterion applied to the one or more movement parameters.

3. The method of claim 2,
wherein the identifying includes identifying one or more areas within the image frames that contain at least one of a hand or arm of the subject;
wherein the determining one or more movement parameters includes determining one or both of amplitude and frequency of movement of the at least one of the hand or arm; and
wherein the generating the indication of the symptom of Parkinson's Disease includes generating an indication of a tremor of the subject according to the determined one or both of amplitude and frequency.

4. The method of claim 3, including changing, by the processing circuitry, a parameter of neurostimulation provided to the subject according to the determined one or both of amplitude and frequency of movement.

5. The method of claim 4, including changing, by the processing circuitry, one or both of a target amplitude and a target frequency bandwidth of movement of the at least one of the hand or arm.

6. The method of claim 2,
wherein the identifying includes identifying an area within the image frames that contains eyes of the subject;
wherein the determining one or more movement parameters includes determining frequency of eye blinking of the subject; and
wherein the generating the indication of the symptom of Parkinson's Disease includes generating an indication of a change in the frequency of the eye blinking as the symptom of Parkinson's disease.

7. The method of claim 2,
wherein the identifying includes identifying an area within the image frames that contains eyes of the subject;
wherein the determining one or more movement parameters includes detecting saccadic eye movement of the subject; and
wherein the generating the indication of the symptom of Parkinson's Disease includes generating an indication of abnormal eye movement as the symptom of Parkinson's disease.

8. The method of claim 7, including changing, by the processing circuitry, a parameter of neurostimulation provided to the subject in response to the detected abnormal eye movement.

9. The method of claim 2,
wherein the identifying includes identifying one or more areas within the image frames that contain at least one limb of the subject;
wherein the determining one or more movement parameters includes determining one or more of limb position and direction of limb movement of the at least one limb; and
wherein the generating the indication of the symptom of Parkinson's Disease includes generating an indication of abnormality in gait of the subject according to one or more detection criteria applied to the determined one or more of limb position and direction of limb movement.

10. The method of claim 9, including changing, by the processing circuitry, a parameter of neurostimulation provided to the subject in response to the detected gait abnormality.

11. The method of claim 2,
wherein the identifying includes identifying an area within the image frames that contains a thorax of the subject;
wherein the determining one or more movement parameters includes determining one or both of amplitude and frequency of movement of the thorax; and
wherein the generating the indication of the symptom of Parkinson's Disease includes generating an indication of change in respiratory rate of the subject according to the determined one or both of amplitude and frequency.

12. The method of claim 2,
wherein the identifying includes identifying an area within the image frames that contain a facial image of the subject;
wherein the analyzing the video data includes analyzing the video data of the facial image to detect a change in appearance of the facial image between a first frame of video data and a later frame of video data;
wherein the determining the one or more change parameters from the video data includes detecting one or more of facial rigidity, a change in pupil dilation, and a change in skin pigment; and
wherein the generating the indication of a symptom of Parkinson's disease includes generating an indication of a symptom of Parkinson's Disease according to a detection criterion applied to the one or more change parameters.

13. The method of claim 12, including changing, by the processing circuitry, a parameter of neurostimulation provided to the subject in response to the detected change in one or more of facial rigidity, pupil dilation, and skin pigment.

14. The method of claim 1, wherein obtaining a video stream of the subject using a video data source includes obtaining the video stream using a device to provide prompts to a user while obtaining the video stream.

15. An electronic device, including:
a video data source configured to obtain a video stream that includes image frames of video data;
processing circuitry; and
a memory storing instructions that, when performed by the processing circuitry, cause the processing circuitry to perform operations comprising:
identifying one or more areas within the image frames of the video stream that contain a physiological feature of a subject;
analyzing video data of the identified one or more areas in the image frames to detect movement of the physiological feature between a first frame of video data and a later frame of video data;
determining one or more movement parameters of the at least one physiological feature from the video data; and
generating an indication of a tremor associated with Parkinson's Disease according to a detection criterion applied to the one or more movement parameters.

16. The electronic device of claim 15, including instructions that cause the processing circuitry to perform operations including:
identifying one or more areas within the image frames that contain at least one of a hand or arm of the subject;
determining one or both of amplitude and frequency of movement of the at least one of the hand or arm; and
generating an indication of a tremor of the subject according to the determined one or both of amplitude and frequency.

17. The electronic device of claim 15, including instructions that cause the processing circuitry to perform operations including:
identifying one or more areas within the image frames that contain at least one limb of the subject;
determining one or more of limb position and direction of limb movement of the at least one limb; and
generating an indication of abnormality in gait of the subject according to one or more detection criteria applied to the determined one or more of limb position and direction of limb movement.

18. The electronic device of claim 15, including instructions that cause the processing circuitry to change one or more parameters of neurostimulation therapy provided to the subject by a separate device according to the determined one or more movement parameters.

19. An electronic device, including:
a video data source configured to obtain a video stream that includes image frames of video data;
processing circuitry; and
a memory storing instructions that, when performed by the processing circuitry, cause the processing circuitry to perform operations comprising:
identifying one or more areas within the image frames of the video stream that contain a facial image of a subject;
analyzing video data of the identified one or more areas in the image frames to detect a change in appearance of the facial image between a first frame of video data and a later frame of video data;
detecting one or more of facial rigidity, a change in pupil dilation, and a change in skin pigment from the video data; and
generating an indication of a symptom of Parkinson's Disease according to a detection criterion applied to the detected one or more of facial rigidity, change in pupil dilation, and change in skin pigment.

20. The electronic device of claim 19, including instructions that cause the processing circuitry to change one or more parameters of neurostimulation therapy provided to the subject by a separate device according to the detected change in the one or more of facial rigidity, pupil dilation, and skin pigment.
